(19) United States
(12) Patent Application Publication
(10) Pub. No.: US 2019/0242863 A1
(43) Pub. Date: Aug. 8, 2019

(54) RELEASE TEST SYSTEM FOR SIMULATING THE CHANGE IN STATE OF MEDICAL ACTIVE INGREDIENTS

(71) Applicant: PHYSIOLUTION GMBH, Greifswald (DE)

(72) Inventors: Grzegorz GARBACZ, Greifswald (DE); Anne DEUTER, Levenhagen (DE); Olga FRONCZYK, Greifswald (DE); Grzegorz DOMANSKI, Greifswald (DE); Harald BELOW, Greifswald (DE); Romy BAGUHL, Greifswald (DE)

(73) Assignee: PHYSIOLUTION GMBH, Greifswald (DE)

(21) Appl. No.: 16/342,657

(22) PCT Filed: Oct. 17, 2017
(86) PCT No.: PCT/EP2017/076403

§ 371 Date: Apr. 17, 2019

(30) Foreign Application Priority Data
Oct. 20, 2016 (DE) 10 2016 120 019.0

(51) Int. Cl. G01N 33/15; G01N 13/00

(52) U.S. Cl. CPC G01N 33/15; G01N 2013/006; G01N 13/00

(57) ABSTRACT

The invention relates to a release test system (10) for simulating the change in state of medical active ingredients in the region of a human or animal organ, comprising at least:
a main body (12) of a release test vessel (16);
a lid (14) of the release test vessel (16);
technical control means (18) for influencing simulation conditions prevailing within the release test vessel (16), wherein the technical control means (18) comprise at least one stirring mechanism (20) and one temperature control device (22); and further comprising
a sampling device (26).

Provision is made for the lid (14) and at least the stirring mechanism (20) to be permanently connected to one another and for a nondetachable connection to be producible or to exist between the main body (12) and the lid (14).

The present invention relates to a release test system according to the preamble of claim 1.

One important aspect to be considered when developing medical active ingredients is how a medical active ingredient is taken up by the human body. One important influencing factor in this respect is the state of the medical active ingredient or of a given dosage form of the medical active ingredient and its change in the human body. Dosage forms may, for example, assume liquid form from the outset. They may, however, also initially assume solid form, then be present as a liquid-solid mixture, and finally adopt liquid form or complete dissolved form. As a rule, a chain of states of the dosage form, before it can be ultimately absorbed by the body, is longer for solid dosage forms than for dosage forms that are liquid from the outset. This is associated with the fact that solid dosage forms generally have to be finely comminuted and dissolved before absorption by the body. Within the human body, these operations take place, for example, via the masticatory apparatus or through certain conditions inside the human body which lead to a change in state of the medical active ingredients. Examples of this are mechanical, physical and chemical influences in the region of the gastrointestinal passage. A change in the state of the medical active ingredient is brought about as a result of influencing factors prevailing there, such as temperature, presence of body fluids, pH, presence of gases, pressure conditions, presence of further solids and mechanical influences resulting from muscle contractions of the stomach and intestine. This change in state may, for example, be a change from a solid state in the form of a tablet, through finely comminuted fragments of the tablet to the state of being dissolved in liquid.

Since the state of the medical active ingredient at the absorption site in the human body, for example in stomach lining regions, at the gastrointestinal passage or in the intestine, has a major effect on uptake efficiency and thus on the action of the medical active ingredient, medical active ingredients need to be developed in such a way that the change in state thereof in the human body has a defined characteristic which favors uptake. Extensive laboratory investigations are needed in this case, for example to investigate the release behavior of a dosage form in a test liquid.

WO 2013/164629 A1 for example describes a device for testing solubility of a medical dosage form. This device comprises a chamber in which a solvent medium is located. The device comprises means for adjusting pH.

In this case, there are particular challenges when handling the medical active ingredients.

For instance, medical active ingredients may cause undesired toxic, carcinogenic, mutagenic, fertility-compromising or other potentially hazardous effects. This constitutes a risk to health when handling the active ingredients.

It is therefore necessary to prevent the medical active ingredient from exiting a test system and entering the surrounding environment. Conventionally, these challenges are countered by personnel wearing suitable protective clothing and by providing complex environmental monitoring systems and isolated safe working areas.

All this entails significant effort with regard to the technical, organizational and personal protective measures which need to be taken.

The object of the invention is to provide a novel test system for medical active ingredients which can be produced and used with little effort and at the same time is extremely reliable with regard to working conditions and attainable test results.

The object is achieved by the subject matter of independent claim 1. Preferred configurations of the invention are revealed by the remaining features mentioned in the subclaims.

The subject matter of the invention is a release test system for simulating the change in state of medical active ingredients in the region of a human or animal organ, comprising at least:
a main body of a release test vessel;
a lid of the release test vessel;
technical control means for influencing simulation conditions prevailing within the release test vessel, wherein the technical control means comprise at least one stirring mechanism and a temperature control device; and furthermore
a sampling device.

According to the invention, the lid and at least the stirring mechanism are permanently connected to one another and a nondetachable connection is producible or exists between the main body and the lid.

Inert materials are preferably used for the components of the release test system which may come into contact with medical active ingredients during use. For example, stainless steel may be used, but preferably a plastics material is used as it is inexpensive and easy to work. The lid and the main body are connected or connectable together in such a way that once they have been assembled they can no longer be non-destructively separated from one another. The lid and the main body are then integral components of the release test vessel. The lid and the stirring mechanism are preferably designed as a common unit, further preferably can no longer be disassembled once the lid has been connected with the main body, and further preferably can no longer be non-destructively disassembled once the lid and the stirring mechanism have been fitted together. The release test system as a whole is, due to its structure, particularly suitable for one-off use or as a disposable system.

The release test system according to the invention has the advantage that it is of very simple construction and can be produced with little effort. This is also attributable to the fact that no possibility of disassembly has to be structurally provided, thereby increasing design latitude. The release test system is also particularly safe, since after preparation for the simulation, i.e. after filling of the main body with a test liquid, adjusting the temperature of the test liquid and equipping the main body with the dosage form of the medical active ingredient to be tested, followed by fitting of the lid to the main body, such that the release test vessel is permanently closed, no access to the interior of the release test vessel by releasing the lid is possible any longer. Once the simulation is concluded, the release test vessel or the main body including lid may be disposed of as a unit, meaning that there is no possibility of contaminated material escaping. Conventional systems are not suitable for disposable use due to the cost thereof and therefore must be cleaned, which is a complex and high-risk process. In the case of the present invention, such use is ruled out by the design measures taken.

The release test system is particularly well suited for investigating the dissolution behavior of medical active ingredients under conditions such as those that prevail in the region of the human gastrointestinal passage.

In a preferred configuration of the present invention, provision is made for the lid to be form-lockingly and/or force-lockingly connectable or connected and/or materially bondable or bonded to the main body.

The main body and the lid may preferably be connected to one another by way of a snap-in connection, a press fit, an adhesive bond or ultrasound welding. In this case, a snap-in connection, as an example of a form-locking or form- and force-locking connection, offers the advantage that the release test vessel may initially be delivered in the open state, i.e. without a connection present between the main body and the lid, and then permanently closed at the place of use.

The same is true of an adhesive bond as an example of a flexibly producible bonded connection, this having particular advantages with regard to tightness.

A press fit, as an example of a force-locking connection, offers the advantage that the main body and the lid may be permanently and extremely tightly connected to one another without additional parts or auxiliary substances having to be provided for this purpose. This reduces the design complexity of the release test system.

Ultrasound welding, as a further example of a bonded connection, offers significantly increased tightness, durability and security against undesired separation of the lid from the main body.

In view of the technical teaching disclosed here, a person skilled in the art is henceforth in a position to select further suitable connecting methods.

In a further preferred configuration of the release test system of the invention, provision is made for the lid to be hermetically sealed or sealable relative to the main body. Suitable sealing elements are preferably used in this respect. Flat gaskets, sealing rings and indeed sealing pastes are particularly suitable.

All of this offers several advantages at once. On the one hand, security against contaminated material escaping is significantly increased. On the other hand, defined pressure conditions may in this way be produced inside the release test vessel, for example by the technical control means, to influence the simulation conditions prevailing inside the release test vessel. A vacuum may for example be produced here. This ensures that even in the case of undesired leaks in the release test vessel, no contaminated material can escape. However, an overpressure may also be produced, in order for example to simulate gas accumulation in the stomach.

In a further preferred configuration of the release test system of the invention, provision is made for the stirring mechanism to have a drive interface, which is only accessible from outside the release test vessel. The drive interface may for example comprise a shaft end of a drive shaft of the stirring mechanism. The shaft end may be manufactured to fit or indeed have a parallel key or teeth. The shaft end conveniently projects out of the lid of the release test vessel, wherein the lid may comprise appropriate mechanical interfaces for mounting an electrical drive, for example. The drive shaft is preferably firmly installed in the lid via rolling bearings or plain bearings.

The structure described furthermore reduces the design complexity of the release test system and ensures simple and extremely reliable operability from outside. It is additionally advantageously ensured that the stirring mechanism is always arranged at the same position inside the release test vessel. If, for example, series of simulations are conveniently carried out with different release test systems intended for one-off use, the results may in this way be better compared with one another.

The stirring mechanism may particularly preferably be incorporated into the lid in such a way that it is not released with a view to performing a stirring movement until the permanent connection has been produced between the lid and the main body. To this end, for example, a locking element may be provided which, upon closure of the main body with the lid, is displaced relative to the stirring mechanism from a locking position into a release position.

The locking element may ensure that use of the stirring mechanism is only actually possible when the lid is closed. In this way, improper handling may be ruled out. In practice, it has been known for safety aspects to take a back seat under high cost pressures. The possibility cannot therefore be ruled out of attempts being made to use a system repeatedly which is intended for single use. The locking element reliably prevents such attempts.

The locking element is preferably designed in such a way that the drive interface of the stirring mechanism becomes unusable (purely by way of example through a predetermined breaking point in the drive shaft) if an attempt is made to force the stirring mechanism to start up when the lid is open (i.e. with the locking element in the locking position). In this way, it is ensured that the function of the locking element cannot be bypassed even by applying force.

In a further preferred configuration of the release test system of the invention, provision is made for the sampling device to comprise at least one sampling tube or additionally a sample return tube, these each being arrangeable or arranged in a complementary receptacle in the lid. The respective complementary receptacle is preferably configured such that in no spatial position of the release test vessel can contaminated material escape. The sampling tube and optionally sample return tube may preferably be guided in the lid via tight-fitting plain bearings. The plain bearings may for example be regions of resilient material, through which the sampling tube or sample return tube can be guided with resilient deformation of the material. Purely by way of example, rubber inserts may be provided for this purpose. However, a plurality of sealing rings arranged one behind the other may also be provided. A design requirement for the complementary receptacle is full tightness of the release test vessel even when the entire liquid column of the test liquid located inside the release test vessel and of the medical active ingredient dissolved wholly or partly therein acts on the complementary receptacle. A further safety factor may optionally be added in this case, taking account of overpressure producible in the release test vessel. In light of the technical teaching disclosed here, a person skilled in the art is henceforth in a position to select further suitable sealing methods.

All this has the advantage that a sample may be taken from the release test vessel with little effort. To this end, for example, a hose exposed to a vacuum may be slipped onto the sampling tube. To equalize pressures, the corresponding quantity of air may flow in through the sample return tube. Alternatively, recirculation of a sample which has been taken is also possible in this way.

The sampling tube may also be of a telescopic design. For example, an inner tube may be guided displaceably in an outer tube. Any remaining play between the inner tube and the outer tube may preferably be sealed by a lubricant.

This has the advantage that a small defined sample may be collected by short immersion in the test liquid. Capillary action may be exploited in this respect. It is technically more demanding to collect a sample of very small volume by suction, for example using a pump.

In a further preferred configuration of the release test system of the invention, provision is made for one or more of the following features to be incorporated structurally into the stirring mechanism: a feed path for liquid and/or gaseous substances; technical means for measuring pH; technical means for measuring temperature.

Incorporation into the stirring mechanism first of all offers the advantage that available structural space is utilized effectively and the stated technical means may be directly brought up to the test liquid inside the release test vessel by the stirring mechanism. Feed paths are configured and optionally sealed in such a way that test liquid is always prevented from escaping from the inside of the release test vessel. The feed paths may for example be twisted on themselves in such a way that test liquid cannot flow out for that reason alone. The feed paths may also be of appropriately thin construction. Furthermore, the previously described sealing methods may be used.

The feed path has the advantage that titrimetric substances may for example be guided into the release test vessel.

The technical means for measuring pH and temperature may furthermore advantageously be used to regulate the simulation conditions prevailing inside the release test vessel.

In a further preferred configuration of the release test system of the invention, provision is made for a resilient flask to be arranged or arrangeable inside the release test vessel, said being supplied or suppliable with a pressure medium.

This has the advantage that a wide range of pressure conditions may be simulated inside the release test vessel. In particular, biorelevant pressure conditions may be realistically modeled. The intensity and frequency of pressure waves producible with the resilient flask are advantageously suitable for a close-to-reality simulation of pressure conditions at the human gastrointestinal passage.

In a further preferred configuration of the release test system of the invention, provision is made for an auxiliary substance for transferring mechanical stirring energy to the test liquid to be introduced or introducible into the release test vessel. The auxiliary substance may for example be polystyrene pellets.

The auxiliary substance advantageously brings about an increase in the mechanical loading of the dosage form of the medical active ingredient inside the release test vessel. By adapting a test liquid used and a stirring program, it is possible to simulate postprandial conditions for taking rapid-release dosage forms.

In a further preferred configuration of the release test system of the invention, provision is made for the lid to have one or more predetermined breaking points for producing interfaces in the release test vessel, wherein in the region of the interfaces sealing elements or receptacles are provided for this purpose.

Depending on the configuration of the release test system, for example as a function of technical means arranged inside the release test vessel, such as the resilient flask, structural weaknesses are advantageously produced in the lid only when actually necessary. Otherwise, the lid is materially impermeable in the region of the predetermined breaking points, thereby offering maximum safety. The sealing elements or receptacles present may optionally be configured such that they are adaptable with little effort. Purely by way of example, a sealing element in the form of a flat semi-finished product of rubber may be provided, which is perforated if need be with a needle whose diameter is conveniently smaller than for example the diameter of a sampling tube to be placed in the sealing element.

This makes the release test system extremely flexibly configurable and safe at the same time. Since only a one-off configuration is necessary, due to the disposable nature, such simply configured regions may be implemented for producing the interfaces.

In a further preferred configuration of the release test system of the invention, provision is made for the release test system to further comprise one or more of the following features: a drive for the stirring mechanism; a pump for delivering test liquid from and/or into the release test vessel; an analyzer for analyzing the test liquid; one or more stoppers for closing interfaces of the lid.

All this has the advantage of enabling the release test system to offer a simple, reliable and extensively automatable simulation of the change of state of the medical active ingredients located inside the release test vessel. The pump and the analyzer further have the particular advantage that medical active ingredients which have passed into solution may be recirculated with simultaneous analysis. To this end, using the pump test liquid is sucked out of the release test vessel for example via a sampling tube, passed through the analyzer, analyzed and finally passed by way of the pump output through the sample return tube back into the release test vessel. In this way, test runs of extended duration may also advantageously be implemented, in order to observe the change in state of the medical active ingredients continuously over time.

Provided nothing is stated to the contrary, the various features disclosed in this patent application can be combined with one another.

The invention is described in greater detail below with reference to exemplary embodiments and associated figures, in which:

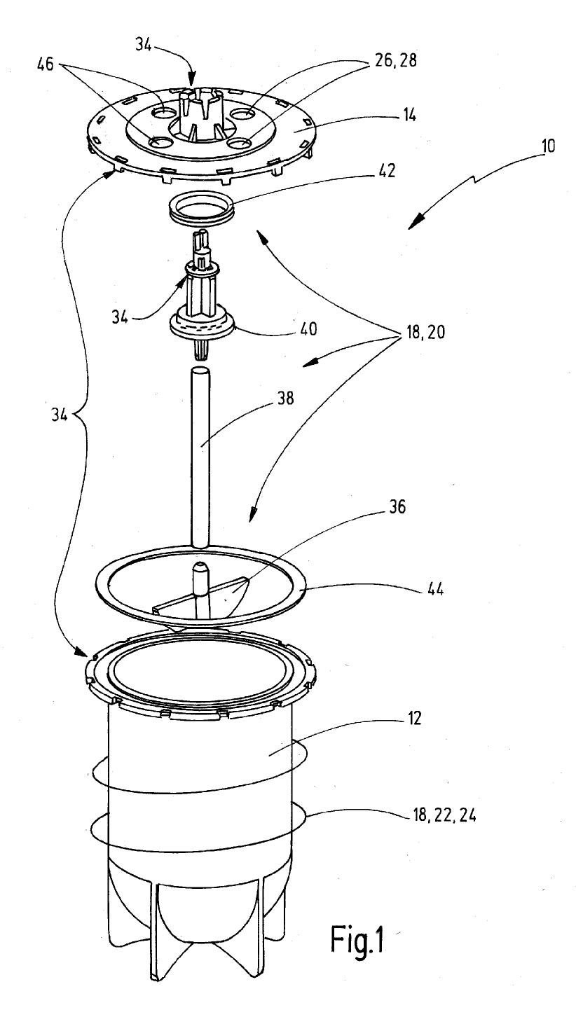

FIG. 1 shows a preferred embodiment of a release test system according to the invention, in an exploded view;

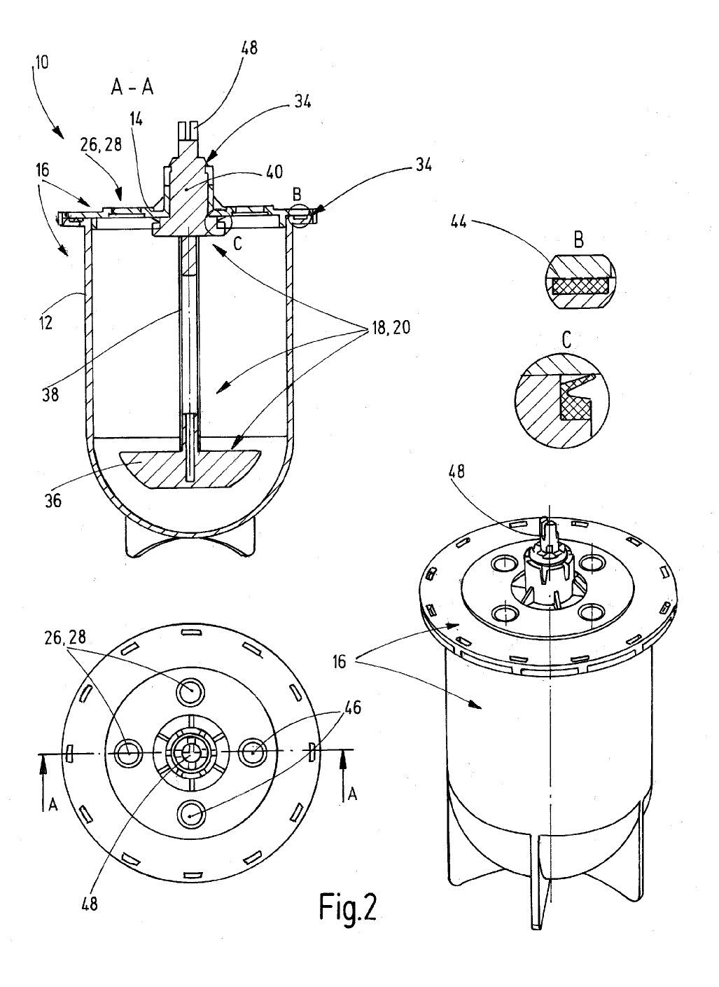

FIG. 2 shows the release test system of FIG. 1 in a cross-sectional view, a plan view and an isometric view;

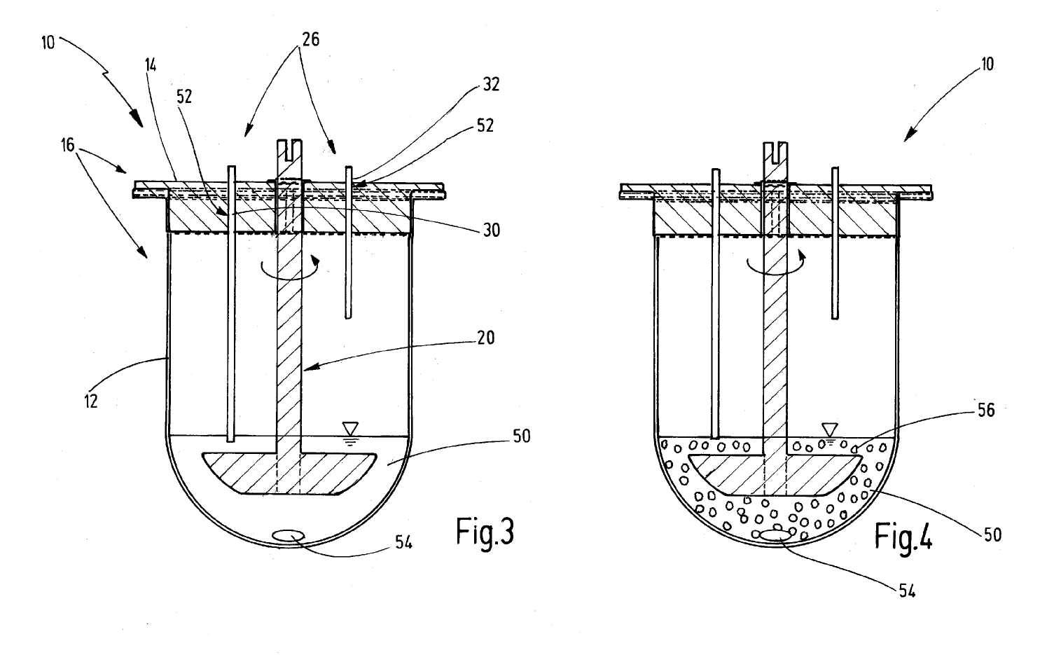

FIG. 3 shows a further preferred embodiment of a release test system according to the invention;

FIG. 4 shows a further preferred embodiment of a release test system according to the invention;

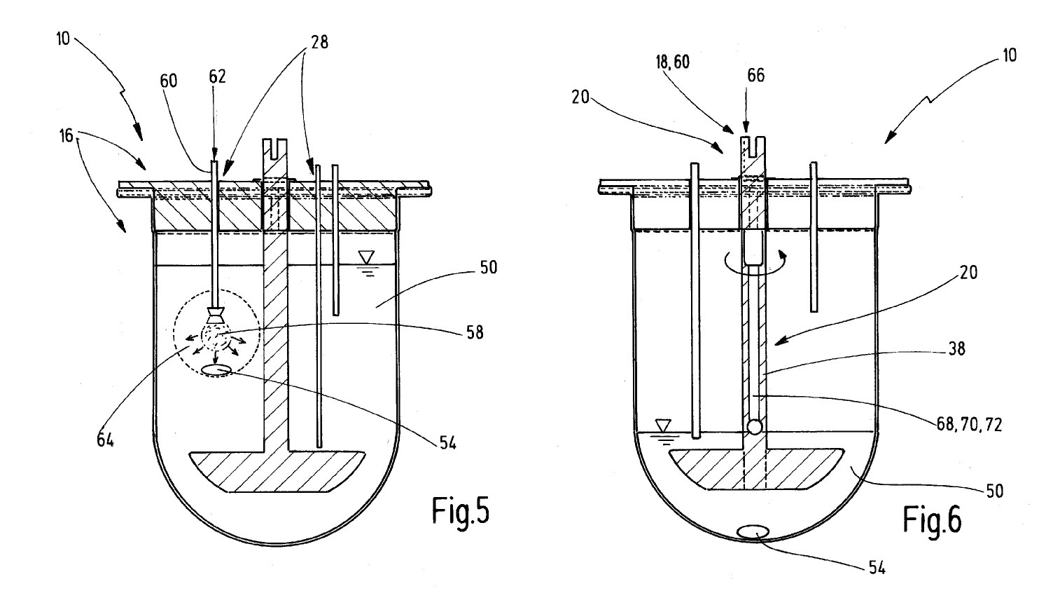

FIG. 5 shows a further preferred embodiment of a release test system according to the invention;

FIG. 6 shows a further preferred embodiment of a release test system according to the invention; and

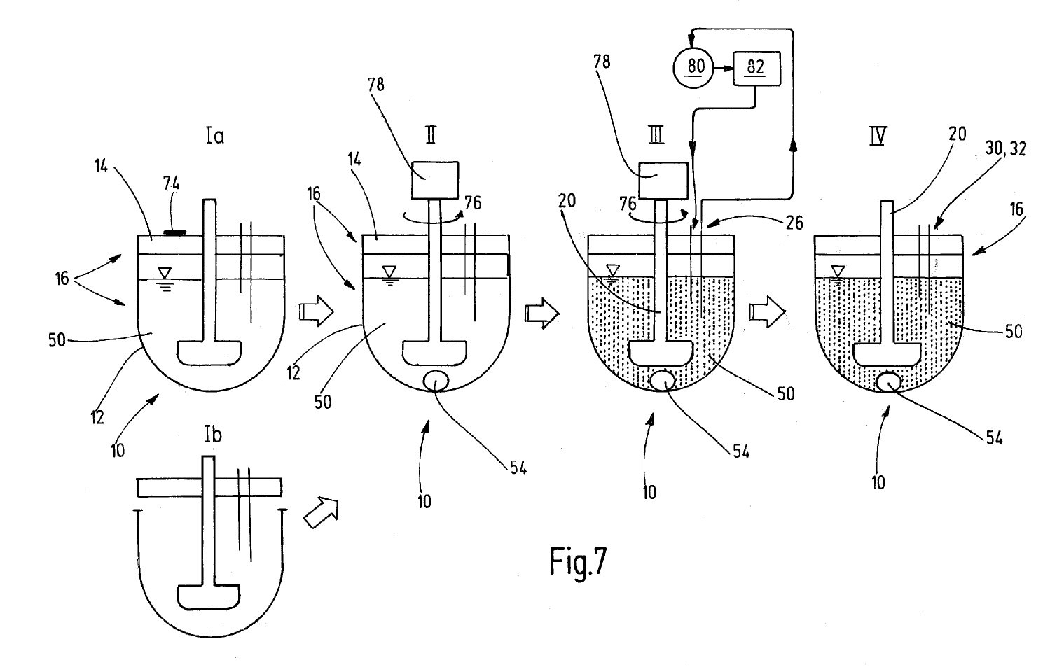

FIG. 7 shows a simulation procedure using a preferred embodiment of a release test system according to the invention.

FIG. 1 shows a preferred embodiment of a release test system 10 according to the invention. The release test system 10 is shown in exploded view. The release test system 10 comprises a main body 12 and a lid 14. The main body 12 and the lid 14 are assemblable into a release test vessel 16 (cf. FIG. 2). The release test system 10 further comprises technical control means 18 for influencing simulation conditions prevailing inside the release test vessel 16. The technical control means 18 comprise at least one stirring mechanism 20 and one temperature control device 22. The temperature control device 22 is shown here purely by way of example as a heating coil 24 on the outside of the main body 12. It may however just as possibly be incorporated into the release test vessel 16 or implemented in another known manner. The release test system 10 further comprises a sampling device 26. The sampling device 26 is implemented in FIG. 1 merely in the form of interfaces 28 which are configured to receive a sampling tube 30 and a sample return tube 32 (cf. in each case FIGS. 3 to 7).

The lid 14 shown in FIG. 1 is connectable with the main body 12 in such a way that the lid 14 and the main body 12 are no longer non-destructively detachable from one another once the connection has been produced. To this end, the lid 14 and the main body 12 have regions for producing a snap-in connection 34, i.e. depending on structural configuration, a form- or additionally force-locking connection. These regions for producing a snap-in connection 34 are shown in yet more detail in FIG. 2. They are configured in such a way that, after assembly of the lid 14 and the main body 12, they can no longer be accessed and the connection can no longer be undone from outside.

The stirring mechanism 20 is incorporated structurally into the lid 14 and thus permanently connected therewith. This mechanism comprises in the present case a paddle stirrer 36, a stirring shaft 38, a bearing portion 40 and a sealing ring 42 arrangeable on the bearing portion 40 and in the lid 14. The bearing portion 40 likewise has in an upper region one of the regions for producing a snap-in connection 34 (cf. FIG. 2). In the present case, the stirring mechanism 20 may thus be simply snapped into the lid 14 and sealed relative thereto with the sealing ring 42.

For hermetic sealing of the lid 14 relative to the main body 12 and of the release test vessel 16 relative to the environment, a flat seal 44 is provided in the region of the snap-in connection. Furthermore, the sealing ring 42 described participates in the hermetic sealing. The interfaces 28 are sealed materially impermeably, provided no sampling device 26 or other required additional technical elements are arranged there. In this case, predetermined breaking points 46 are merely provided in the material of the lid 14. If the interfaces 28 are not needed, the lid 14 is thus itself hermetically impermeable. The seal in the case of use of the interfaces 28 is described further below.

FIG. 2 shows the release test system 10 of FIG. 1 in an assembled state. The release test vessel 16 is shown top left in FIG. 2 in a cross-sectional view A-A with lid 14 mounted on the main body 12. The associated section line A is shown in the lower left-hand part of FIG. 2, in which the release test system 10 is shown in plan view. In the lower right-hand part of FIG. 2, the assembled release test system 10 is shown in an isometric view.

In FIG. 2 the lid 14 and the main body 12 are inseparably connected to one another. A drive interface 48, which projects from the lid 14 and is thus readily accessible from outside, is readily apparent. Likewise easily visible is the arrangement of the flat seal 44 between lid 14 and main body 12. This is shown in detail B. Detail C shows the arrangement of the sealing ring 42 between bearing portion 40 and lid 14. In cross-sectional view A-A the stirring shaft 38 is visible. This is configured in the present case as a hollow shaft. Additional structural space is thus available in which, where needed, technical control means 18 or indeed measuring means may be incorporated (cf. FIG. 6).

FIG. 3 shows a further preferred embodiment of the release test system 10. Indicated therein is how the sampling device 26 with the sampling tube 30 and the sample return tube 32 projects through the lid 14 into the release test vessel 16. The sampling tube 30 in this case projects as far as into a test liquid 50 located in the release test vessel 16. The interfaces 28 known from FIGS. 1 and 2 are here in each case further developed into complementary receptacles 52 in relation to the sampling tube 30 or the sample return tube 32. To this end, for example, the predetermined breaking points 46 in question may have been opened. Different variants are conceivable as to how the exposed interfaces 28 may be further developed into the complementary receptacles 52. For example, sealing elements or receptacles may be provided to this end from the outset under the predetermined breaking points 46. Plain bearings may also be provided in the lid, for example in the form of regions of resilient material which at the same time function as a sealing element. Further development into the complementary receptacles 52 may for example proceed through resilient deformation. A plurality of sealing rings arranged one behind the other may for example be arranged which are expanded when the sampling tube 30 or sample return tube 32 is passed through. It is however also possible to penetrate the resilient regions. It is likewise possible to insert the plain bearings, for example resilient elements, only subsequently.

FIG. 1 shows that a dosage form 54 of a medical active ingredient has been introduced into the test liquid 50. This is by way of example a tablet or capsule.

Through rotation of the stirring mechanism 20, simulation conditions may be mechanically influenced in the release test vessel 16. The temperature control device 22, which is not illustrated here but which may be arranged in or on the release test vessel 16 or indeed separately therefrom, allows the temperature of the test liquid 50 to be adjusted before and/or during and optionally after the simulation.

The sampling device 26 allows simple sampling and recirculation. To this end, either the sample may be taken using the sampling tube 30 and air or another medium added using the sample return tube 32 or indeed the sample taken may be recirculated via the sample return tube 32.

FIG. 4 shows a further preferred embodiment of the release test system 10. Here, an auxiliary substance 56 for transferring mechanical stirring energy to the test liquid 50 and thus the dosage form 54 is additionally introduced into the test liquid 50. The auxiliary substance 56 is by way of example polystyrene pellets.

FIG. 5 shows a further preferred embodiment of the release test system 10. Here a resilient flask 58 is arranged inside the release test vessel 16. Via a feed path 60, which is embodied by way of an appropriately adapted interface of the interfaces 28, the resilient flask 58 may be supplied with a pressure medium 62. The resilient flask 58 is arranged in a sample chamber 64 arranged inside the release test vessel 16, wherein the sample chamber 64 is connected fluidically with the remaining volume located inside the release test vessel 16. The dosage form 54 is arranged in the sample chamber and is thus surrounded by the test liquid 50. This embodiment makes it possible for the dosage form 54 to be exposed purposefully to a pressure or pressure profile characteristic of the relevant human or animal organ. The pressure may be transferred to the dosage form 54 as a pressure wave through the test liquid 50 or through contact with the resilient flask 58.

FIG. 6 shows a further preferred embodiment of the release test system 10. In this embodiment the feed path 60 is incorporated directly into the stirring mechanism 20. The feed path 60 may be configured to guide substances 66 into the release test vessel 16 or to remove them in a controlled manner. The feed path is a further example of a technical control means 18, as is the auxiliary substance 56 of FIG. 4 and the resilient flask 58 of FIG. 5. The substances 66 may be liquid, solid, gaseous or a mixture. Titrimetric substances, carbon dioxide or nitrogen may for example be supplied. Technical means for measuring pH 68 or technical means for measuring temperature 70 may also be incorporated into the stirring mechanism 20. In the present case, for example, an integral pH/temperature sensor 72 is incorporated into the stirring shaft 38.

Finally, FIG. 7 shows by way of example how the release test system 10 according to the invention is in principle to be used. In a step I the release test vessel 16 is provided. The lid 14 and the main body 12 may be delivered, as shown in step Iₐ, already in the preassembled state or, as shown in step I_{b}, in the unassembled state. The unassembled state makes filling of the main body 12 with test liquid 50 simpler, while the preassembled state simplifies sterile transport. If, on the other hand, the main body 12 is preassembled with the lid 14, the release test vessel 16 may be filled via the interfaces 28. These may then be securely closed for example with one or more stoppers 74.

In step II at the latest, filling with test liquid 50 proceeds, and placement of the dosage form, then, at the latest, fitting of the lid 14 onto the main body 12, followed by adjustment of the temperature of the test liquid 50.

In step III at the latest, stirring 76 proceeds with the stirring mechanism 20. To this end, a corresponding drive 78 is connected to the stirring mechanism 20. A sample is further taken with the sampling device 26 via a pump 80, for example, and the sample is analyzed using an analyzer 82. As indicated, recirculation of the sample may optionally take place, to perform a continuous test.

Step IV involves disposal of the release test vessel 16 including stirring mechanism 20, sampling and return tubes and the test liquid 50 and the dosage form 54. In this way, all contaminated elements, which are complex and risky to clean, are directly disposed of. The contaminated test liquid 50 in this case remains safely contained inside the release test vessel 16.

### REFERENCE SIGNS

10 Release test system
12 Main body
14 Lid
16 Release test vessel
18 Technical control means
20 Stirring mechanism
22 Temperature control device
24 Heating coil
26 Sampling device
28 Interfaces
30 Sampling tube
32 Sample return tube
34 Regions for producing a snap-in connection
36 Paddle stirrer
38 Stirring shaft
40 Bearing portion
42 Sealing ring
44 Flat seal
46 Predetermined breaking points
48 Drive interface
50 Test liquid
52 Complementary receptacles
54 Dosage form
56 Auxiliary substance
58 Feed path
60 Resilient flask
62 Pressure medium
64 Sample chamber
66 Substances
68 Technical means for measuring pH
70 Technical means for measuring temperature
72 Integral pH/temperature sensor
74 Stopper
76 Stirring
78 Drive
80 Pump
82 Analyzer

1. A release test system for simulating the change in state of medical active ingredients in the region of a human or animal organ, comprising at least:
a main body of a release test vessel;
a lid of the release test vessel;
technical control means for influencing simulation conditions prevailing within the release test vessel, wherein the technical control means comprise at least one stirring mechanism and one temperature control device; and furthermore
a sampling device;
wherein
the lid and at least the stirring mechanism are permanently connected to one another and a nondetachable connection is producible or exists between the main body and the lid.

2. The release test system according to claim 1, wherein
the lid is form-lockingly and/or force-lockingly connectable or connected and/or is materially bondable or bonded to the main body.

3. The release test system according to claim 1,
wherein
the lid is hermetically sealed or sealable relative to the main body.

4. The release test system according to claim 1,
wherein
the stirring mechanism has a drive interface, which is accessible from outside the release test vessel.

5. The release test system according claim 1,
wherein
the sampling device comprises at least one sampling tube or additionally a sample return tube, these each being arrangeable or arranged in a complementary receptacle in the lid.

6. The release test system according to claim 1,
wherein
one or more of the following features are incorporated structurally into the stirring mechanism: a feed path for liquid and/or gaseous substances; technical means for measuring pH; technical means for measuring temperature.

7. The release test system according to claim 1,
wherein
a resilient flask is arranged or arrangeable inside the release test vessel and can be supplied with a pressure medium.

8. The release test system according to claim 1,
wherein
an auxiliary substance for transferring mechanical stirring energy to a test liquid is introduced or introducible into the release test vessel.

9. The release test system according to claim 1,
wherein
the lid has one or more predetermined breaking points for producing interfaces in the release test vessel, wherein in the region of the interfaces sealing elements or receptacles are provided for this purpose.

10. The release test system according to claim 1,
wherein
the release test system additionally comprises one or more of the following features: a drive for the stirring mechanism; a pump for delivering test liquid from and/or into the release test vessel; an analyzer for analyzing the test liquid; one or more stoppers for closing interfaces of the lid.
